(19) United States
(12) Patent Application Publication
(10) Pub. No.: US 2026/0183204 A1
(43) Pub. Date: Jul. 2, 2026

(54) OIL-IN-WATER EMULSION COMPOSITION

(71) Applicant: SHISEIDO COMPANY, LTD., Tokyo (JP)

(72) Inventor: Shizuma TANAKA, Tokyo (JP)

(73) Assignee: SHISEIDO COMPANY, LTD., Tokyo (JP)

(21) Appl. No.: 19/417,517

(22) Filed: Dec. 12, 2025

(30) Foreign Application Priority Data
Dec. 27, 2024 (JP) 2024-232350

(51) Int. Cl. A61K 8/06; A61K 8/67; A61K 8/81; A61Q 19/00

(52) U.S. Cl. CPC A61K 8/062; A61K 8/671; A61K 8/8147; A61Q 19/00; A61K 2800/5422

(57) ABSTRACT

The oil-in-water emulsion composition of the present invention suppresses the denaturation of a retinol derivative, even in severe environments such as high temperatures. The present invention relates to an oil-in-water emulsion composition comprising: (A) 0.01 to 4% by mass of a retinol derivative; (B) 0.05 to 3% by mass of powder made from a highly water-absorbent polymer having an absorption ratio of 10 or more times its own weight; (C) an oil excluding higher alcohols which are solid at normal temperature; (D) water; and (E) a nonionic surfactant.

### RELATED APPLICATIONS

This application claims priority from Japanese Patent Application No. 2024-232350 filed on Dec. 27, 2024. The entire teachings of the above application are incorporated herein by reference.

### BACKGROUND OF THE INVENTION

### Field of the Invention

The present invention relates to an oil-in-water emulsion composition. More specifically, the present invention relates to an oil-in-water emulsion composition that can stably retain a retinol derivative, and a cosmetic made of the composition.

### Description of the Related Art

Retinols (vitamin A) and retinol derivatives are known as components effective for the prevention or treatment of skin keratosis and the like or for the prevention or recovery of skin aging, for example, and have heretofore been blended into cosmetics or topical skin preparations.

The retinols are also known to be unstable and tend to comparatively easily denature due to a certain influence such as light, air, heat, or metal ions. Retinol fatty acid ester, which is a retinol derivative, is superior in oxidative stability to retinol. However, since the retinol fatty acid ester might undergo hydrolysis, it is necessary to carefully formulate an oil-in-water emulsion system for securing over-time stability of the retinol fatty acid ester.

For example, JP-A Hei 11-228377 discloses an oil-in-water emulsion composition comprising a vitamin A fatty acid ester, an oil-soluble antioxidant, an amphiphilic substance, and a hydrophilic nonionic surfactant. In this document, it is necessary, for improving the over-time stability of the vitamin A fatty acid ester, to set the transition temperature of a gel formed in an amphiphilic substance-hydrophilic nonionic surfactant-water system to 50° C. or higher and to adjust the weight ratio between the amphiphilic substance and the hydrophilic nonionic surfactant and the total amount of the surfactants to predetermined ranges.

JP-B 4406035 discloses an oil-in-water emulsion skin cosmetic comprising a vitamin A and a gel formed in the same manner as in Japanese JP-A Hei 11-228377. According to this document, to stabilize vitamin A and improve the texture of the cosmetic, the amount of a higher alcohol with 14 to 24 carbon atoms should be limited to a specific range, and a combination of three types of nonionic surfactants should be used.

JP B 4330511 discloses an oil-in-water topical skin preparation in which a vitamin A fatty acid ester is encapsulated in an oil particle surrounded by a shell made of a specific amphiphilic substance. In this topical skin preparation, the vitamin A fatty acid ester is contained within the oil particle, preventing the vitamin A fatty acid ester from coming into contact with water and improving the stability of the vitamin A fatty acid ester.

The techniques described in JP A Hei 11-228377, JP-B 4406035 and JP-B 4330511 could improve the stability of retinol (vitamin A) and its derivatives to some extent. However, they were sometimes insufficient for preventing the denaturation of retinol derivatives in severe conditions such as high-temperature environment. Additionally, the conventional techniques mentioned above require precise adjustments to the types and amounts of amphiphilic substances and nonionic surfactants, which limits the range of possible prescriptions.

Therefore, it is earnestly required to provide a technic further improve stably of a retinol derivative over the conventional techniques, and to provide an oil-in-water emulsion composition applicable to various prescriptions.

### SUMMARY OF THE INVENTION

The present inventor has conducted diligent studies and consequently completed the present invention by finding that an effect of stably retaining a retinol derivative can be further improved by blending a powder made from a highly water-absorbent polymer into an oil-in-water emulsion composition containing the retinol derivative.

Specifically, the present invention provides an oil-in-water emulsion composition comprising:
(A) 0.01 to 4% by mass of a retinol derivative;
(B) 0.05 to 3% by mass of powder made from a highly water-absorbent polymer having an absorption ratio of 10 or more times its own weight;
(C) an oil excluding higher alcohols which are solid at normal temperature;
(D) water; and
(E) a nonionic surfactant.

The oil-in-water emulsion composition of the present invention suppresses the denaturation of a retinol derivative, even in severe environments such as high temperatures. This effect is achieved by blending a powder made from a highly water-absorbent polymer, making it applicable to a wide range of formulations.

### DETAILED DESCRIPTION OF THE INVENTION

The oil-in-water emulsion composition (hereinafter, also simply referred to as the “composition”) of the present invention comprises: (A) a retinol derivative; (B) a highly water-absorbent polymer powder having a water absorption ratio of 10 or more times its own weight; (C) an oil (excluding higher alcohols which are solid at normal temperature); (D) water; and (E) a nonionic surfactant as essential components.

### (A) Retinol Derivative

The retinol derivative (also referred to as the “component (A)”) according to the present invention includes fatty acid ester of retinol (vitamin A), retinal (vitamin A aldehyde), retinoic acid (vitamin A acid), and their salts. In the present invention, retinol fatty acid ester or a salt thereof is preferably used as the component (A). Examples of the retinol fatty acid ester include, but are not limited to, retinol acetate, retinol palmitate, retinol propionate, and retinol linoleate. Examples of salt include alkali metal salts (e.g., sodium salt, potassium salt, and lithium salt), alkaline earth metal salts (e.g., calcium salt and magnesium salt), ammonium salts, and organic amine salts (e.g., monoethanolamine salt, diethanolamine salt, and triethanolamine salt).

The amount of the retinol derivative (A) in the composition of the present invention is 0.01 to 4% by mass, preferably 0.05 to 2% by mass, based on the total amount of an oil-in-water emulsion cosmetic. A concentration of less than 0.01% by mass of the retinol derivative is insufficient to exert its effect, while a concentration of more than 4% by mass may overstimulate the skin.

(B) Powder Made from Highly Water-Absorbent Polymer

The powder made from highly water-absorbent polymer powder (also referred to as the “highly water-absorbent polymer powder” or the “component (B)”) according to the present invention is a powder consisting of a highly water-absorbent polymer having an absorption ratio of 10 or more times its own weight. The “water absorption ratio” as used herein is a numeric value indicating that the weight of water absorbed by the polymer powder corresponds to how many times its own weight of the polymer powder which can be measured by various measurement methods (e.g., a dry weight method and the like) applicable to the highly water-absorbent polymer powder.

The highly water-absorbent polymer powder (B) according to the present invention is preferably a powder that contains a cross-linked acrylic polymer structure and has a globular shape, preferably a spherical shape, in a dry state. The particle size of the highly water-absorbent polymer powder (B) in the oil-in-water emulsion composition is not particularly limited, and a powder having an average particle size of usually 1 to 50 μm, preferably 1 to 30 μm, is used. One kind of highly water-absorbent polymer powder (B) may be used, or two or more kinds of highly water-absorbent polymer powders (B) may be blended as a mixture.

The water absorption ratio of the highly water-absorbent polymer powder (B) should be 10 or more times. The highly water-absorbent polymer powder (B) used in this disclosure has the water absorption ratio of preferably 50 or more times, more preferably 100 or more times. As used herein, a powder having a water absorption ratio of 100 or more times is referred to as an “ultra-highly water-absorbent polymer powder” and may be described distinctively from a highly water-absorbent polymer powder having a water absorption ratio of less than 100 times.

Specific examples of the highly water-absorbent polymer powder (B) can include powders consisting of a salt of cross-linked acrylic acid polymer. Particularly, a powder consisting of a polymer designated as “sodium acrylates crosspolymer-2” (INCI name) is preferred. Commercially available products of the powder consisting of sodium acrylates crosspolymer-2 can be used in this invention. Examples thereof can include ARON NT-Z (manufactured by Toagosei Co., Ltd.). This commercially available product is known to have a water absorption ratio of approximately 24 times.

Preferred examples of the highly water-absorbent polymer powder (B) belonging to the ultra-highly water-absorbent polymer powder include powders consisting of a polymer designated as “sodium carbomer” (INCI name), and powders consisting of a polymer designated as “sodium polyacrylate starch” (INCI name). AQUPEC MG N40R (manufactured by Sumitomo Seika Chemicals Co., Ltd.) is known as a commercially available product of “sodium carbomer”, and MAKIMOUSSE 12 (average particle size: approximately 12 μm) (manufactured by Daito Kasei Kogyo Co., Ltd.), MAKIMOUSSE 25 (average particle size: approximately 25 μm) (manufactured by Daito Kasei Kogyo Co., Ltd.), SANFRESH ST-100 (manufactured by Sanyo Chemical Industries, Ltd.), and the like are known as commercially available products of “sodium polyacrylate starch”. Also, AQUAKEEP 10SH-NF (manufactured by Sumitomo Seika Chemicals Co., Ltd.) is also a powder consisting of “sodium acrylates crosspolymer-2” (INCI name) but can be used as the ultra-highly water-absorbent polymer powder because its water absorption ratio is 200 to 500 times. In the composition of the present invention, an ultra-highly water-absorbent polymer powder consisting of sodium carbomer is preferably used as the highly water-absorbent polymer powder (B).

The amount of the highly water-absorbent polymer powder (B) in the composition of the present invention is 0.05 to 3% by mass, preferably 0.1 to 2% by mass, based on the total amount of the composition. In the case of using an ultra-highly water-absorbent polymer powder as the highly water-absorbent polymer powder (B), the range of the amount of the ultra-highly water-absorbent polymer powder is set to preferably 0.2 to 1% by mass, and more preferably 0.25 to 0.7% by mass. The highly water-absorbent polymer powder (B) in an amount of less than 0.05% by mass is insufficient for stabilizing the retinol derivative.

### (C) Oil

The composition of the present invention is an oil-in-water emulsion composition. The oil (also referred to as the “component (C)”) constituting the emulsion composition is not particularly limited and includes (C1) a liquid oil and (C2) a solid or semisolid oil that are usually used in cosmetics, medicaments, and the like. However, the (C) oil does not include higher alcohols which are solid at normal temperature (25° C.). In this disclosure, “higher alcohol” means monohydric alcohols having six or more carbon atoms.

The liquid oil (also referred to as the “component (C1)”) is an oil that is a liquid at normal temperature (25° C.) (including oils having a melting point of lower than 2° C.). Specific examples thereof include: liquid fats and oils such as avocado oil, evening primrose oil, camellia oil, macadamia nut oil, sunflower oil, almond oil, corn oil, olive oil, rapeseed oil, egg yolk oil, sesame oil, apricot kernel oil, wheat germ oil, sasanqua oil, castor oil, flaxseed oil, safflower oil, cottonseed oil, perilla oil, soybean oil, peanut oil, tea seed oil, kaya oil, rice bran oil, paulownia oil, Japan tung oil, jojoba oil, and germ oil; ester oils such as cetyl octanoate, cetyl ethylhexanoate, hexyldecyl dimethyloctanoate, ethyl laurate, hexyl laurate, isopropyl myristate, 2-hexyldecyl myristate, myristyl myristate, octyldodecyl myristate, isopropyl palmitate, 2-ethylhexyl palmitate, 2-hexyldecyl palmitate, 2-heptylundecyl palmitate, butyl stearate, isocetyl stearate, isocetyl isostearate, decyl oleate, dodecyl oleate, oleyl oleate, myristyl lactate, cetyl lactate, diisostearyl malate, cholesteryl 12-hydroxystearate, castor oil fatty acid methyl ester, N-lauroyl-L-glutamic acid-2-octyldodecyl ester, 2-ethylhexyl succinate, diisobutyl adipate, 2-hexyldecyl adipate, di-2-heptylundecyl adipate, diisopropyl sebacate, di-2-ethylhexyl sebacate, cetyl ethylhexanoate, ethylene glycol di-2-ethylhexanoate, neopentyl glycol dicaprate, neopentyl glycol dioctanoate, acetoglyceride, glycerin di-2-heptylundecanoate, tricaprylin, glycerin trioctanoate, glycerin tri-2-ethylhexanoate, glycerin trimyristate, glycerin triisopalmitate, glyceride tri-2-heptylundecanoate, trimethylolpropane tri-2-ethylhexanoate, trimethylolpropane triisostearate, pentaerythrityl tetraoctanoate, pentaerythrityl tetraethylhexanoate, and di(phytosteryl/octyldodecyl) lauroyl glutamate; hydrocarbon oils such as liquid paraffin, ozokerite, squalane, pristane, polybutene, hydrogenated polybutene, and hydrogenated polydecene; higher alcohols which are liquid at normal temperature, such as isostearic alcohol, and oleil alcohol; silicone oils including linear chain polysiloxane such as dimethylpolysiloxane (dimethicone), methylphenylpolysiloxane, and diphenylpolysiloxane, cyclic polysiloxane such as octamethylcyclotetrasiloxane, decamethylcyclopentasiloxane, and dodecamethylcyclohexasiloxane, and modified polysiloxane such as amino-modified polysiloxane, polyether-modified polysiloxane, alkyl-modified polysiloxane, and fluorine-modified polysiloxane.

The solid or semisolid oil (also referred to as the “component (C2)”) is an oil that is a solid or a paste at normal temperature (25° C.) (including oils having a melting point of 25° C. or higher). The component (C2) does not include higher alcohols. Specific examples of component (C2) include: solid fats and oils such as cacao butter, coconut oil, hydrogenated coconut oil, palm oil, hydrogenated palm oil, palm kernel oil, Japan wax kernel oil, Japan wax, and hydrogenated castor oil; waxes such as beeswax, candelilla wax, cotton wax, carnauba wax, bayberry wax, privet wax, montan wax, bran wax, lanolin, kapok wax, lanolin acetate, sugar cane wax, lanolin fatty acid isopropyl ester, hexyl laurate, reduced lanolin, jojoba wax, hard lanolin, shellac wax, POE lanolin alcohol ether, POE lanolin alcohol acetate, POE cholesterol ether, and POE hydrogenated lanolin alcohol ether; hydrocarbon waxes such as polyethylene wax, paraffin wax, ceresin, petrolatum, microcrystalline wax, lunacera, and ozokerite; fatty acid glyceryl ether such as monostearyl glycerin ether (batyl alcohol); and fatty acid glyceride such as acetoglyceride and glyceride tri-2-heptylundecanoate; and fatty acid sterol esters such as macadamia nut fatty acid phytosteryl.

The amount of the oil (C) in the composition of the present invention is preferably 8% by mass or more, more preferably 12% by mass or more, furthermore preferably 18% by mass or more based on the total amount of the composition. The lower limit of the amount of component (C) is not particularly limited to these and may be, for example, 10% by mass or more, 16% by mass or more, or 20% by mass or more based on the total amount of the composition. An increase in the amount of oil (C) was found to improve the stability of retinol derivatives. The upper limit of the amount of the oil (C) is not particularly limited, and the upper limit can be, for example, 50% by mass or less, 40% by mass or less, 35% by mass or less, or 30% by mass or less based on the total amount of the composition.

The oil (C) in the composition of the present invention preferably contains at least 60%, more preferably at least 70%, and most preferably at least 80%, by mass, of the (C1) liquid oil, based on the total amount of oil (C).

In the composition of the present invention, the oil (C) preferably contains the (C2) solid or semisolid oil. When the composition contains (C2) solid or semisolid oil, it can comprise 1% by mass or more, preferably 2% by mass or more, and no more than 20% by mass, preferably no more than 18% by mass, and more preferably no more than 15% by mass, based on the total amount of oil (C).

### (D) Water

The amount of water (also referred to as the “component (D)”) in the composition of the present invention is 80% by mass or less, preferably 70% by mass or less, more preferably 60% by mass or less, based on the total amount of the composition. In the present invention, blending a smaller amount of water (D) tends to improve the stabilizing effect on the retinol derivative.

### (E) Nonionic Surfactant

The oil-in-water emulsion composition of the present invention contains a nonionic surfactant (also referred to as the “component (E)”).

Any of a “hydrophilic nonionic surfactant” having an HLB value of 12 or more and a “lipophilic nonionic surfactant” having an HLB value of less than 12 can be used as the nonionic surfactant (E) according to the present invention. The HLB (hydrophile-lipophile balance) value of each substance can be determined in accordance with the Kawakami method or the Griffin method. In the Kawakami method, the HLB value is calculated on the basis of the following expression.

$$HLB = 7 + 11.7 log ( Mw / Mo )$$

wherein Mw represents the molecular weight of a hydrophilic group, and Mo represents the molecular weight of a lipophilic group.

Examples of the hydrophilic nonionic surfactant can include: polyoxyethylene (hereinafter, abbreviated to “POE”) alkyl ethers (including POE behenyl ether, POE oleyl ether, POE stearyl ether, POE 2-octyl dodecyl ether, POE 2-hexyl decyl ether, POE 2-heptyl undecyl ether, POE 2-decyl tetradecyl ether, POE2-decyl pentadecyl ether, POE cholestanol ether, etc.); POE glycerin fatty acid esters (including POE glyceryl stearate, POE glyceryl isostearate, etc.); POE sorbitan fatty acid esters (including POE sorbitan monooleate, etc.); and POE hydrogenated castor oils. More specifically, examples thereof can include POE (20) behenyl ether (beheneth-20), PEG-100 stearate, PEG-60 glyceryl isostearate, and PEG-60 hydrogenated castor oil.

Examples of the lipophilic nonionic surfactant include fatty acid glycerin esters and POE glycerin fatty acid esters. More specifically, examples thereof include glyceryl stearate (HLB=5 to 6), self-emulsifying (SE) glyceryl stearate (HLB=5 to 8), glyceryl isostearate (HLB=6), and PEG-5 glyceryl stearate (HLB=9).

The amount of the nonionic surfactant (E) in the composition of the present invention can be 0.1% by mass or more, 0.2% by mass or more, 0.3% by mass or more, 0.4% by mass or more, or 0.5% by mass or more, and 5% by mass or less, 4% by mass or less, 3% by mass or less, 2% by mass or less, or 1% by mass or less, based on the total amount of the composition. In other words, the nonionic surfactant (E) is preferably blended in an amount that ranges from equal to or more than any of the lower limit values described above to equal to or less than any of the upper limit values described above.

According to the present invention, one nonionic surfactant (E) may be used, or two or more nonionic surfactants (E) may be blended together. At least one hydrophilic nonionic surfactant is preferably included. As for the lipophilic nonionic surfactant, a surfactant having an HLB value of 5 or higher is preferable. Sorbitan tristearate, for example, has an HLB value of 3 or less and is preferably absent or blended in an amount of less than 0.1 in terms of the total mass of the nonionic surfactant (E).

The composition of the present invention preferably further contains (F) an amphiphilic substance (also referred to as the “component (F)”). The amphiphilic substance (F) is preferably selected from the substances capable of forming an a gel in a water phase, together with the nonionic surfactant (E) described above. The a gel is considered as a homogeneous gel state in which a layered structure (a type hydrate crystals) formed from a bimolecular membrane composed of a surfactant and an amphiphilic substance forms a network in a state solubilized in water. The a gel thus formed further improves the emulsification stability of the composition.

The amphiphilic substance (F) in the present invention is a higher alcohol which is solid at normal temperature (25° C.). The higher alcohol which is solid at normal temperature is preferably selected from monohydric higher alcohols having an alkyl group including 16 to 22 carbon atoms. Among those, saturated aliphatic alcohols are preferably used. Specific examples thereof include behenyl alcohol, stearyl alcohol, and cetyl alcohol (cetanol).

The amount of the amphiphilic substance (F) in the composition of the present invention can be 1% by mass or more, 2% by mass or more, or 3% by mass or more, and 10% by mass or less, 8% by mass or less, or 6% by mass or less, based on the total amount of the composition. In other words, the amphiphilic substance (F) is preferably blended in an amount that ranges from equal to or more than any of the lower limit values described above to equal to or less than any of the upper limit values described above.

The ratio (mass ratio) between the amount of the nonionic surfactant (E) and the amount of the amphiphilic substance (F) is preferably adjusted to the range of 10:1 to 1:10, preferably 7:1 to 1:7.

The composition of the present invention may contain, in addition to the components (A) to (F) described above, other components that may be blended into cosmetics or quasi-pharmaceutical products. Examples of other components can include, but are not limited to, thickeners, humectants, various drugs, antioxidants, chelating agents, pH adjusters, ultraviolet absorbers, and powder components.

Examples of the thickener can include aqueous thickeners such as water-soluble acrylic acid polymers and water-soluble polysaccharides. However, the thickener excludes those having a water absorption ratio of 10 or more times.

Examples of the water-soluble acrylic acid polymer can include copolymers of sodium acrylate and acryloyldimethyl taurate, and copolymers of dimethylacrylamide and acryloyldimethyl taurate. Specific examples thereof include (dimethylacrylamide/sodium acryloyldimethyl taurate) crosspolymers, (hydroxyethyl acrylate/sodium acryloyldimethyl taurate) copolymers, and (acrylates/(C10-30)alkyl acrylate) crosspolymers. Examples of the water-soluble polysaccharide include xanthan gum and hydroxyethylcellulose.

Examples of the humectant include polyethylene glycol, propylene glycol, dipropylene glycol (DPG), 1,3-butylene glycol (BG), hexylene glycol, glycerin, diglycerin, xylitol, maltitol, maltose, D-mannitol, starch syrup, glucose, fructose, lactose, sodium chondroitin sulfate, sodium hyaluronate, sodium adenosine phosphate, sodium lactate, bile salt, pyrrolidonecarboxylic acid, glucosamine, and cyclodextrin.

Examples of the various drugs include vitamins other than the retinol derivative (A), skin-lightening agents such as 4-methoxysalicylate and arbutin, amino acids, anti-inflammatory agents, and astringents.

Examples of the antioxidant include oil-soluble antioxidants such as butylhydroxytoluene (BHT), butylhydroxyanisole (BHA), and α,β,γ,δ-tocopherol, and water-soluble antioxidants such as sodium pyrosulfite.

The composition of the present invention can be produced in accordance with a method commonly used for oil-in-water emulsion cosmetics. For example, an emulsion can be prepared by first mixing the aqueous components with stirring, then heating and stirring the oil components separately, and finally adding the oil components to the aqueous components with stirring.

The composition of the present invention thus configured is excellent in emulsification stability and in addition, can stably retain the retinol derivative even under severe conditions.

Thus, the composition of the present invention is suitable for a topical skin preparation or a skin cosmetic having efficacy based on the retinol derivative. The form of the composition provided as the cosmetic is not limited and can be a liquid cosmetic such as a liquid foundation, a skin cream, a body cream, or a face cream.

### Examples

Hereinafter, the present invention will be described in more detail with reference to examples. However, the present invention is not limited by the examples given below. The amount of each component in the following examples refers to % by mass based on the total amount of the composition.

Each oil-in-water emulsion composition was prepared in accordance with the prescription described in Tables 1 to 3 below. The composition of each example was evaluated for the stability of the retinol derivative by the following method.

The composition of each example was placed in a container, shielded from light with aluminum foil, and preserved at 60° C. for 2 weeks. Then, the amount of a residual retinol derivative (retinol acetate) was measured by high-performance liquid chromatography. The residual rate (or residual ratio), of the amount of retinol derivative remaining after preservation, relative to the initial amount blended into each composition, was calculated using the following formula.

$$Residual rate ( \% ) = [ ( Amount after preservation ) / ( Initial Amount ) ] \times 100$$

The calculated residual rates are also described in Tables 1 to 3.

The measurement by high-performance liquid chromatography was performed under the following conditions.
Column: C18 column (manufactured by Shiseido Co., Ltd.)
Detection: UV 310 nm
Mobile phase: 72% methanol/10% acetonitrile/18% ion exchange water/0.5% acetic acid

TABLE 1

| | Comparative | Example | Example | Example | Example |
|---|---|---|---|---|---|
| | Example 1-1 | 1-1 | 1-2 | 1-3 | 1-4 |
| Retinol acetate | 0.1 | 0.1 | 0.1 | 0.1 | 0.1 |
| Highly-water-absorbent polymer | — | 0.1 | 0.3 | 0.5 | 0.7 |
| powder (*) | | | | | |
| Beheneth-20 | 0.7 | 0.7 | 0.7 | 0.7 | 0.7 |
| Behenyl alcohol | 3.3 | 3.3 | 3.3 | 3.3 | 3.3 |
| Stearyl alcohol | 0.9 | 0.9 | 0.9 | 0.9 | 0.9 |
| Macadamia nut fatty acid | 1 | 1 | 1 | 1 | 1 |
| phytosteryl | | | | | |
| Hydrogenated palm oil | 1 | 1 | 1 | 1 | 1 |
| Palm kernel oil | 0.6 | 0.6 | 0.6 | 0.6 | 0.6 |
| Palm oil | 0.4 | 0.4 | 0.4 | 0.4 | 0.4 |
| Squalane | 8 | 8 | 8 | 8 | 8 |
| Pentaerythrityl | 12 | 12 | 12 | 12 | 12 |
| tetraethylhexanoate | | | | | |
| Di(phytosteryl/octyldodecyl) | 1 | 1 | 1 | 1 | 1 |
| lauroyl glutamate | | | | | |
| Dimethicone | 1.5 | 1.5 | 1.5 | 1.5 | 1.5 |
| Glycerin | 5 | 5 | 5 | 5 | 5 |
| BG | 8 | 8 | 8 | 8 | 8 |
| (Dimethylacrylamide/sodium | 0.3 | 0.3 | 0.3 | 0.3 | 0.3 |
| acryloyldimethyl taurate) | | | | | |
| crosspolymer | | | | | |
| Xanthan gum | 0.05 | 0.05 | 0.05 | 0.05 | 0.05 |
| Potassium 4-methoxysalicylate | 1 | 1 | 1 | 1 | 1 |
| Sodium pyrosulfite | 0.02 | 0.02 | 0.02 | 0.02 | 0.02 |
| BHT | 0.05 | 0.05 | 0.05 | 0.05 | 0.05 |
| EDTA-3Na | 0.1 | 0.1 | 0.1 | 0.1 | 0.1 |
| Citric acid | 0.02 | 0.02 | 0.02 | 0.02 | 0.02 |
| Sodium citrate | 0.08 | 0.08 | 0.08 | 0.08 | 0.08 |
| Water | Balance | Balance | Balance | Balance | Balance |
| Total | 100 | 100 | 100 | 100 | 100 |
| Residual rate | 76% | 80% | 85% | 86% | 84% |

(*) Sodium carbomer (AQUPEC MG N40R: manufactured by Sumitomo Seika Chemicals Co., Ltd.)

As shown in Table 1, for Comparative Example 1-1 in which sodium carbomer, the highly water-absorbent polymer powder (B), was absent, the residual rate of the retinol acetate (A) was 76% after preservation at 60° C. for 2 weeks. By contrast, Examples 1-1 to 1-4 comprising the highly water-absorbent polymer powder (B) indicated the residual rate of the retinol acetate of 80% or more even after preservation at 60° C. for 2 weeks. In other words, the addition of the highly water-absorbent polymer powder (B) enabled the retention of 80% or more of retinol acetate (A) during preservation at high temperatures.

TABLE 2

| | Comparative | Example | Example | Example | Example |
|---|---|---|---|---|---|
| | Example 2-1 | 2-1 | 2-2 | 2-3 | 2-4 |
| Retinol acetate | 0.1 | 0.1 | 0.1 | 0.1 | 0.1 |
| Highly-water-absorbent polymer | — | 0.1 | 0.3 | 0.5 | 0.7 |
| powder (*) | | | | | |
| Beheneth-20 | 0.35 | 0.35 | 0.35 | 0.35 | 0.35 |
| Behenyl alcohol | 2.6 | 2.6 | 2.6 | 2.6 | 2.6 |
| Stearyl alcohol | 0.7 | 0.7 | 0.7 | 0.7 | 0.7 |
| Squalane | 3 | 3 | 3 | 3 | 3 |
| Pentaerythrityl | 5 | 5 | 5 | 5 | 5 |
| tetraethylhexanoate | | | | | |
| Dimethicone | 2 | 2 | 2 | 2 | 2 |
| Glycerin | 5 | 5 | 5 | 5 | 5 |
| BG | 8 | 8 | 8 | 8 | 8 |
| (Dimethylacrylamide/sodium | 0.3 | 0.3 | 0.3 | 0.3 | 0.3 |
| acryloyldimethyl taurate) | | | | | |
| crosspolymer | | | | | |
| Xanthan gum | 0.1 | 0.1 | 0.1 | 0.1 | 0.1 |
| Potassium 4-methoxysalicylate | 1 | 1 | 1 | 1 | 1 |
| Sodium pyrosulfite | 0.02 | 0.02 | 0.02 | 0.02 | 0.02 |
| BHT | 0.05 | 0.05 | 0.05 | 0.05 | 0.05 |
| EDTA-3Na | 0.1 | 0.1 | 0.1 | 0.1 | 0.1 |
| Citric acid | 0.02 | 0.02 | 0.02 | 0.02 | 0.02 |
| Sodium citrate | 0.08 | 0.08 | 0.08 | 0.08 | 0.08 |
| Water | Balance | Balance | Balance | Balance | Balance |
| Total | 100 | 100 | 100 | 100 | 100 |
| Residual rate | 52% | 55% | 61% | 63% | 63% |

(*) Sodium carbomer (AQUPEC MG N40R: manufactured by Sumitomo Seika Chemicals Co., Ltd.)

TABLE 3

| | Comparative | Example | Example | Example | Example |
|---|---|---|---|---|---|
| | Example 3-1 | 3-1 | 3-2 | 3-3 | 3-4 |
| Retinol acetate | 0.1 | 0.1 | 0.1 | 0.1 | 0.1 |
| Highly-water-absorbent polymer | — | 0.1 | 0.3 | 0.5 | 0.7 |
| powder (*1) | | | | | |
| PEG-100 stearate (*2) | 1.5 | 1.5 | 1.5 | 1.5 | 1.5 |
| Glycerol stearate (SE) (*3) | 2.5 | 2.5 | 2.5 | 2.5 | 2.5 |
| Behenyl alcohol | 0.85 | 0.85 | 0.85 | 0.85 | 0.85 |
| Stearyl alcohol | 0.55 | 0.55 | 0.55 | 0.55 | 0.55 |
| Cetanol | 4 | 4 | 4 | 4 | 4 |
| Squalane | 3 | 3 | 3 | 3 | 3 |
| Pentaerythrityl | 10 | 10 | 10 | 10 | 10 |
| tetraethylhexanoate | | | | | |
| Dimethicone | 3 | 3 | 3 | 3 | 3 |
| Glycerin | 7 | 7 | 7 | 7 | 7 |
| BG | 5 | 5 | 5 | 5 | 5 |
| DPG | 5 | 5 | 5 | 5 | 5 |
| (Dimethylacrylamide/sodium | 0.6 | 0.6 | 0.6 | 0.6 | 0.6 |
| acryloyldimethyl taurate) | | | | | |
| crosspolymer | | | | | |
| Xanthan gum | 0.1 | 0.1 | 0.1 | 0.1 | 0.1 |
| Sodium pyrosulfite | 0.02 | 0.02 | 0.02 | 0.02 | 0.02 |
| BHT | 0.05 | 0.05 | 0.05 | 0.05 | 0.05 |
| EDTA-3Na | 0.05 | 0.05 | 0.05 | 0.05 | 0.05 |
| Citric acid | 0.01 | 0.01 | 0.01 | 0.01 | 0.01 |
| Sodium citrate | 0.09 | 0.09 | 0.09 | 0.09 | 0.09 |
| Phenoxyethanol | 0.5 | 0.5 | 0.5 | 0.5 | 0.5 |
| Water | Balance | Balance | Balance | Balance | Balance |
| Total | 100 | 100 | 100 | 100 | 100 |
| Residual rate | 51% | 58% | 65% | 68% | 66% |

(*1) Sodium carbomer (AQUPEC MG N40R: manufactured by Sumitomo Seika Chemicals Co., Ltd.)
(*2) PEG-100 stearate (HLB = 19)
(*3) Glyceryl stearate (SE) (HLB = 5.5)

The compositions described in Table 1 contain 22.5% by mass of the (C1) liquid oils and 3.0% by mass of the (C2) solid or semisolid oils. In contrast, the compositions described in Tables 2 and 3 contain only (C1) liquid oils and the amounts thereof are 10.0% by mass (Table 2) and 16.0% by mass (Table 3). For the compositions in Tables 2 and 3, the residual rate of retinol acetate (A) decreased to about 50% after preservation at 60° C. for two weeks when the highly water-absorbent polymer powder (B) was absent (Comparative Examples 2-1 and 3-1). Examples 2-1 to 2-4 and Examples 3-1 to 3-4 confirmed that incorporating the highly water-absorbent polymer powder (B) into the compositions notably increased the residual rate of the retinol acetate (A). Additionally, the results in Tables 1 to 3 revealed a tendency for the residual rate of retinol acetate to improve with an increase in the amount of oil (C).

What is claimed is:

1. An oil-in-water emulsion composition comprising:
(A) 0.01 to 4% by mass of a retinol derivative;
(B) 0.05 to 3% by mass of powder made from a highly water-absorbent polymer having an absorption ratio of 10 or more times its own weight;
(C) an oil excluding higher alcohols which are solid at normal temperature;
(D) water; and
(E) a nonionic surfactant.

2. The composition according to claim 1, further comprising:
(F) an amphiphilic substance selected from higher alcohols which are solid at normal temperature.

3. The composition according to claim 1, wherein the retinol derivative (A) is a retinol fatty acid ester.

4. The composition according to claim 1, wherein the highly water-absorbent polymer consists of sodium carbomer.

5. The composition according to claim 1, wherein the nonionic surfactant (E) is a hydrophobic nonionic surfactant having an HLB value of 12 or more.

6. A topical skin preparation consisting of the composition according to claim 1.

7. A skin cosmetic consisting of the composition according to claim 1.
